(19) United States
(12) Patent Application Publication
(10) Pub. No.: US 2008/0215022 A1
(43) Pub. Date: Sep. 4, 2008

(54) Multi-layered protective article

(76) Inventors: Terri Knuth, Antioch, IL (US); Victor Trucker, Antioch, IL (US)

Correspondence Address: LAMPEL & ASSOCIATES, P.C., 555 SKOKIE BLVD., SUITE 500, NORTHBROOK, IL, 60062, US

(21) Appl. No.: 11/712,341

(22) Filed: Mar. 1, 2007

(51) Int. Cl. A61F 13/15

(52) U.S. Cl. 604/359

(57) ABSTRACT

The present invention generally provides a multi-layered protective article designed to be used in, for example, undergarments. The multi-layered protective article may not only create a barrier between the individual and the undergarment, but may also act as an absorbent. Further, the multi-layered protective article may contain a fragrance, a deodorant, lotions and/or medications. The protective article may also have tabs which allow the individual to easily remove a soiled layer to expose the clean under-layer. The protective article allows the individual to extend the life of the undergarment and, therefore, save money.

### BACKGROUND OF THE INVENTION

The present invention generally provides a multi-layered protective article designed to be used in, for example, undergarments. The multi-layered protective article may not only create a barrier between the individual and the undergarment, but may also act as an absorbent. Further, the multi-layered protective article may contain a fragrance, a deodorant, lotions and/or medications. The protective article may also have tabs which allow the individual to easily remove a soiled layer to expose the clean under-layer. The protective article allows the individual to extend the life of the undergarment and, therefore, save money.

Every year millions of undergarments are discarded as a result of discoloration and/or uncleanness. This is especially true of white or other lightly colored undergarments. The discoloration is often a result of the production of sweat, incontinence and/or excessive contact with the skin of the individual. Although washing the undergarments often removes the discoloration from many of the garments, washing is not always effective and/or desirable. Instead, it is common for some of the remaining discoloration and/or odor to remain in the undergarment even after washing. As a result, many individuals elect to discard the undergarments and purchase new undergarments. Therefore, millions of dollars are spent on replacing discolored and/or unclean undergarments with new undergarments.

Replacing old undergarments with new undergarments is not only desirable, but also healthy; however, the protective article of the present invention can greatly decrease the need to constantly purchase new undergarments by increasing the lifespan of undergarments. As a result, a small investment in the present multi-layered protective article may save money by allowing the individual to avoid the cost of repeated replacement of expensive undergarments.

Protective articles designed to protect undergarments have been known and used by people for many years. However, there exists no known article which protects undergarments in the way the present invention protects undergarments. More specifically, there exists no known multi-layered protective article. Further there exists no known multi-layered protective article which has tabs for easily removing each of the layers.

### SUMMARY OF THE INVENTION

The present invention generally provides a multi-layered protective article designed to be used in, for example, undergarments. The multi-layered protective article may not only create a barrier between the individual and the undergarment, but may also act as an absorbent. Further, the multi-layered protective article may contain a fragrance, a deodorant, lotions and/or medications. The protective article may also have tabs which allow the individual to easily remove a soiled layer to expose the clean under-layer. The protective article allows the individual to extend the life of the undergarment and, therefore, save money.

The multi-layered protective article of the present invention is suitable for use by many individuals. For example, undergarments used by athletes are often exposed to heavy sweating, heat and friction when the athlete is running, exercising or engaging in other strenuous activity. As a result, the lifespan of the undergarment of an athlete is often rather short. Providing a multi-layered protective article to protect the undergarment from direct contact with the athlete would greatly increase the lifespan of the undergarment. Further, an athlete may simply remove an individual layer of the protective article to expose a clean layer after the athlete finishes his/her activities. Therefore, the athlete may work out, remove an individual layer and continue on the rest of the day if, for example, the athlete is unable to shower or otherwise clean up after the workout.

In addition to athletes, people taking medications would benefit from the use of the protective article. For example, if an individual is taking a medication to reduce hemorrhoid suffering, the medication would be prevented from soiling the undergarment.

There exists single layered protective articles for use by individuals to protect undergarments, but such articles are limited to a single use. The individual must then remove the individual protective article once soiled and replace the soiled protective article with an entirely new protective article. As a result, the individual might be required to carry numerous protective articles if a change is required during the day. Further, the individual may be required to spend a good deal of money for constantly replacing the single layer protective article.

In the preferred embodiment, a protective article for an article of clothing is provided comprising: a first protective layer having a top surface and a bottom surface; a second protective layer having a top surface and a bottom surface and wherein the bottom surface of the first layer is located substantially above the top surface of the second layer; and an adhesive temporarily securing the bottom surface of the first layer to the top surface of the second layer wherein the first layer may be removed from the second layer.

In another embodiment, the protective article has a tab located on the first and/or second layer wherein the tab allows the individual to grasp and separate the first layer from the second layer.

In yet another embodiment, the protective article has a plurality of layers attached to the second layer.

In still another embodiment, the layers of the protective article may absorb moisture.

And in another embodiment, the layers include a fragrance, lotion or medication.

In an embodiment, the tabs are staggered.

In still another embodiment, indicia is displayed on the tabs.

In an embodiment, the layers are flushable.

For a more complete understanding of the above listed features and advantages of the multi-layered protective article, reference should be made to the following detailed description of the preferred embodiments and to the accompanying drawings. Further, additional features and advantages of the present invention are described in, and will be apparent from, the detailed description of the preferred embodiments and from the drawings.

### BRIEF DESCRIPTION OF THE DRAWINGS

FIG. 1 illustrates a perspective top view of the protective article of the present invention wherein one of the layers is being removed to exposed a clean layer.

FIG. 2 illustrates the bottom layer of the present invention wherein the indicia informing the user of the last layer is visible and wherein the coated layer is visible.

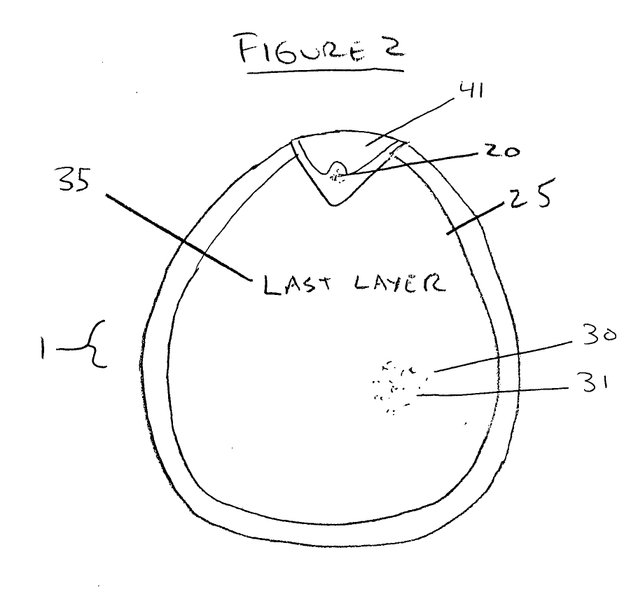

### DETAILED DESCRIPTION OF THE PREFERRED EMBODIMENTS

The present invention generally provides a multi-layered protective article designed to be used in, for example, undergarments. The multi-layered protective article may not only create a barrier between the individual and the undergarment, but may also act as an absorbent. Further, the multi-layered protective article may contain a fragrance, a deodorant, lotions and/or medications. The protective article may also have tabs which allow the individual to easily remove a soiled layer to expose the clean under-layer. The protective article allows the individual to extend the life of the undergarment and, therefore, save money.

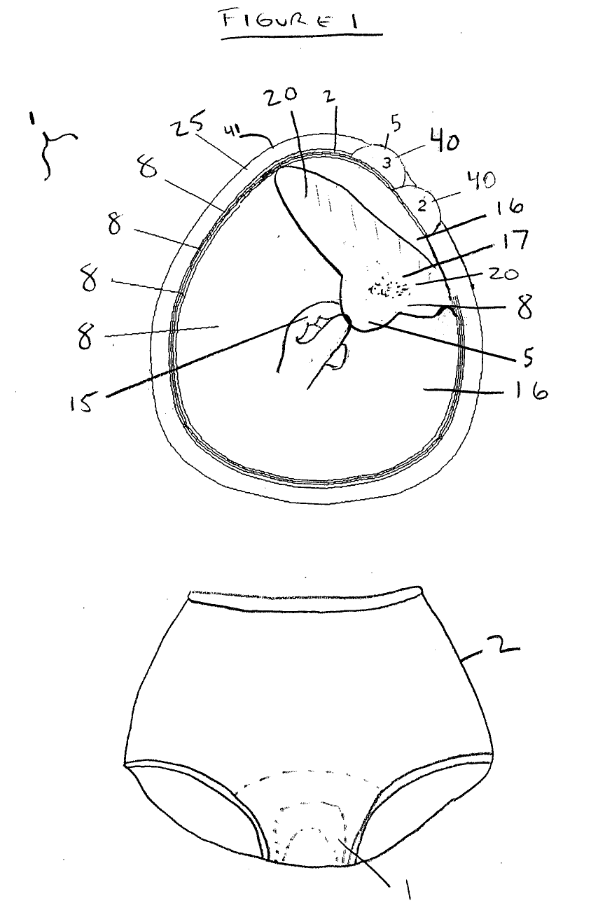

Referring now to the drawings wherein like numerals refer to like parts, FIG. 1 generally illustrates a multi-layered protective article 1 for use in, for example, an undergarment 2. It should be noted that the multi-layered protective article 1 may be used in other clothing articles aside from undergarments. The multi-layered protective article 1 is preferably shaped like a wide teardrop, however, any shape may be implemented to achieve its desired function. The multi-layered protective article 1 may be constructed from cloth, paper or any other suitable material. Preferably, the multi-layered protective article 1 is made from a flexible material so as to provide comfort to the individual. Although the multi-layered protective article 1 may be absorbent, it preferably acts as a barrier between an individual and the undergarment 2. The multi-layered protective article 1 is suitable for use by both males and females.

The individual layers 8 of the multi-layered protective article 1 may each have a top side 16 and a bottom side 17. The top side 16 may be suitable for contact with the skin of the individual. The bottom side 17 of the individual layers 8 of the multi-layered protective article 1 may have a temporary permanent adhesive 20. The adhesive 20 may allow for the bottom side 17 of one of the individual layers 8 to be loosely attached to the top side 16 of the individual layer 8 directly beneath it. More specifically, the adhesive 20 should prevent the individual layers 8 from moving with respect to one another; however, when an individual layer 8 is removed from the multi-layered protective article 1 it is desirable to have the adhesive 20 substantially remain on the bottom side 17 of the removed individual layer 8 and little or no adhesive 20 remnants present on the top side 16 of the now exposed individual layer 8.

The multi-layered protective article 1 may have a tab 5 attached to each of the layers 10. Preferably, the tab 5 extends from a side of each of the individual layers 8. The tabs 5 may be generally the shape of, for example, a semi-circle or a square and may be large enough to be easily grasped by the individual's fingers 15. There may be no adhesive 20 on either side of the multiple tabs 5. Further, the tabs 5 may have indicia 40 such as, for example, numbers, letters or words, so as to indicate to the individual how many layers remain in the multi-layered protective article 1. Preferably, the multiple tabs 5 are soft so as not to aggravate the skin of the individual.

As the name indicates, the multi-layered protective article 1 may have numerous individual layers 8. The very bottom layer 25 of the multi-layered protective article 1 may have a more permanent adhesive than the adhesive 20 used between the individual layers 8. Therefore, the multi-layered protective article 1 may be more securely attached to the undergarment 2 and at the same time the more permanent adhesive will not come into contact with the top side 16 of any of the individual layers 8. The very bottom layer may be attached to a coated layer 41, made from, for example, wax, plastic, copolymers, olefins, nylons, polyester, polyethylene or polypropylene, which prevents the more permanent layer 25 for sticking to an unwanted surface prior to use. The individual may remove the coated layer 41 prior to inserting the multi-layered protective article 1 into the undergarment 2. Further, the top side 16 of the very bottom-layer 25 of the multi-layered protective article 1 may have, for example, indicia 35 and/or may be colored differently from the individual layer 8 immediately above. The indicia 35 and/or the different coloration may provide information to the individual that there are no individual layers 8 beneath the very bottom layer 25. As a result, the individual would know to replace the multi-layered protective article 1 in the near future.

The individual layers 8 of the multi-layered protective article 1 may have, for example, a fragrance 30 and/or a deodorant 31. The fragrance 30 and/or deodorant 31 may, obviously, reduce unpleasant odor as a result of direct contact with the individual. Further, the individual layer 8 may have a pre-applied ointment such as, for example a medication and/or lotion. The individual may elect to remove one of the individual layers 8 when that specific individual layer 8 becomes discolored or otherwise soiled. As a result, the immediate individual layer 8 beneath the removed layer is now exposed and the individual may be in direct contact with a clean, sterile layer. The removed individual layer 8 would then be discarded, preferably by flushing in a toilet.

Although embodiments of the present invention are shown and described therein, it should be understood that various changes and modifications to the presently preferred embodiments will be apparent to those skilled in the art. Such changes and modifications may be made without departing from the spirit and scope of the present invention and without diminishing its attendant advantages. It is, therefore, intended that such changes and modifications be covered by the appended claims.

1. A protective article for an article of clothing comprising:
a first protective layer having a top surface and a bottom surface;
a second protective layer having a top surface and a bottom surface and wherein the bottom surface of the first layer is located substantially above the top surface of the second layer;
an adhesive temporarily securing the bottom surface of the first layer to the top surface of the second layer wherein the first layer may be removed from the second layer;
a plurality of tabs located on the first and second layer wherein the tabs allow an individual to grasp and separate the first layer from the second layer and wherein the plurality of tabs are staggered: and
indicia located on the tabs wherein the indicia indicates the number of layers remaining in the protective article.

2. The protective article of claim 1 further comprising:
a tab located on the first and/or second layer wherein the tab allows an individual to grasp and separate the first layer from the second layer.

3. The protective article of claim 1 further comprising:
a plurality of layers attached to the second layer.

4. The protective article of claim 3 wherein the layers may absorb moisture.

5. The protective article of claim 1 wherein the layers include a fragrance, lotion or medication.

6. The protective article of claim 2 wherein the tabs are staggered.

7. The protective article of claim 2 wherein the tabs display indicia.

8. The protective article of claim 1 wherein the layers are flushable.
